(19) United States
(12) Patent Application Publication
(10) Pub. No.: US 2024/0285849 A1
(43) Pub. Date: Aug. 29, 2024

(54) COMBINED SENSOR AND INFUSION SYSTEM

(71) Applicants: Katherine Wolfe, Dunwoody, GA (US); Bella Nava, Los Angeles, CA (US); Benjamin Pagliuso, Marina Del Rey, CA (US)

(72) Inventors: Katherine Wolfe, Dunwoody, GA (US); Bella Nava, Los Angeles, CA (US); Benjamin Pagliuso, Marina Del Rey, CA (US)

(73) Assignee: PercuSense, Inc., Valencia, CA (US)

(21) Appl. No.: 18/590,847

(22) Filed: Feb. 28, 2024

Related U.S. Application Data
(60) Provisional application No. 63/449,025, filed on Feb. 28, 2023.

(51) Int. Cl. A61M 5/142; A61M 5/14; A61M 5/158; A61M 5/172

(52) U.S. Cl. CPC A61M 5/14244; A61M 5/1413; A61M 5/158; A61M 2005/1726

(57) ABSTRACT

In one embodiment a system to monitor an analyte and dispense an infusate is disclosed. The system includes an on-body assembly with a first piece and a second piece. The first piece contains a sensor and a cannula and the cannula is in fluid communication with a first piece fluid channel. The second piece contains a pump mechanism connected to a second piece fluid channel. When the first piece is coupled to the second piece, the first piece fluid channel is placed in fluid communication with the second piece fluid channel to establish fluid transport from the pump mechanism through the cannula.

### RELATED APPLICATIONS

This application claims the benefit of U.S. provisional application Ser. No. 63/449,025, filed on Feb. 28, 2023. The application listed above is hereby incorporated by reference in its entirety for all purposes.

### FIELD OF THE INVENTION

The present invention is generally directed to devices and methods that enable a single device to infuse fluids and perform in vivo monitoring of an analyte or analytes such as, but not limited to glucose, lactate, or ketones. In particular, the devices and methods are for infusing fluids such as medicants like insulin into a subject while further monitoring the presence or amount of an analyte or analytes within the subject using an electrochemical sensor.

### BACKGROUND OF THE INVENTION

In vivo monitoring of particular analytes can be critically important to short-term and long-term well being. For example, the monitoring of glucose can be particularly important for people with diabetes in order to determine insulin or glucose requirements. Additionally, the monitoring of multiple analytes associated with metabolic health, such as lactate, can provide additional insight. The need to perform continuous or near continuous monitoring of at least one analyte has resulted in the development of a variety of devices and methods. Some methods place electrochemical sensor devices designed to detect the desired analyte in blood vessels while other methods place the devices in subcutaneous or interstitial fluid.

Commercially available glucose sensors are typically placed in subcutaneous or interstitial fluid and for users, insertion of the sensor involves using an insertion device that uses a needle or sharp to pierce the skin and insert the sensor at a preferred depth below the skin. For many users, there may be a sense of anxiety associated with using the insertion device as any device piercing the skin can potentially draw blood or result in pain or discomfort. An increasing number of people with diabetes also infuse insulin using an insulin pump that requires the subcutaneous placement or insertion of an infusion cannula or catheter. Accordingly, for the population that utilize both an implantable glucose sensor and an infusion pump, two separate insertions are required.

The claimed invention seeks to reduce the number of insertions performed by users of both implantable sensors and infusion devices. With a reduced number of insertions, users may benefit from a reduction in anxiety and a simplified process to garner the benefits of both real-time sensors and infusion devices. In many examples discussed below, one or more analytes may be measured.

### BRIEF SUMMARY OF THE INVENTION

In one embodiment a system to monitor an analyte and dispense an infusate is described. The system includes an on-body assembly made up of a first piece and a second piece. The first piece contains a sensor and a cannula and the cannula is in fluid communication with a first piece fluid channel. The second piece contains a pump mechanism connected to a second piece fluid channel. When the first piece is coupled to the second piece, the first piece fluid channel is placed in fluid communication with the second piece fluid channel to establish fluid transport from the pump mechanism through the cannula.

In another embodiment a system to monitor an analyte and dispense an infusate is described that includes an on-body assembly with a communication module, a processor, a memory and a power supply. The on-body assembly includes a first piece that further has a sensor and a cannula where the cannula is in fluid communication with a first piece fluid channel. The on-body assembly further includes a second piece that has a pump mechanism that is coupled to a second piece fluid channel. When the first piece is coupled to the second piece the first piece fluid channel is placed in fluid communication with the second piece fluid channel to establish fluid communication between the pump mechanism and the cannula.

Other features and advantages of the invention will become apparent from the following detailed description, taken in conjunction with the accompanying drawings that illustrate, by way of example, various features of embodiments of the invention.

### BRIEF DESCRIPTION OF THE DRAWINGS

FIG. 1 is an exemplary block diagram showing components within a system for an on-body assembly that enables a single device to monitor the presence of at least one analyte within a subject while further enabling delivery of an infusate to the subject, in accordance with embodiments of the invention.

FIGS. 2A-2C are exemplary illustrations of an on-body assembly that includes sensor parts and infusion pump parts, in accordance with embodiments of the present invention.

FIGS. 3A and 3B are exemplary illustrations of an alternative on-body assembly that includes sensor parts and infusion pump parts, in accordance with embodiments of the present invention.

FIG. 4 is another exemplary illustration of an on-body assembly having a two piece fluid channel, in accordance with embodiments of the present invention.

FIG. 5 is an exemplary cross-sectional view looking normal to the page, toward the distal ends of a sensor and cannula and a sharp, in accordance with embodiments of the present invention.

FIGS. 6A-6C are exemplary illustrations of an interface between the sharp, the sensor, and the cannula during an insertion process, in accordance with embodiments of the present invention.

### DETAILED DESCRIPTION

Presented below are embodiments of a system that combines an infusion device and sensor system or analyte monitoring system that is intended to enable delivery of an infusate and continuous real-time in-vivo electrochemical sensing of an analyte or molecule, or analytes or molecules of interest within a subject. The system is intended to incorporate an infusion device capable of delivering an infusate to a subject such as, but not limited to insulin. Accordingly, the infusion components may be configured to deliver the infusate within subcutaneous tissue. Similarly, the in-vivo measurement within a subject is typically performed in tissue such as, but not limited to subcutaneous tissue. However, various embodiments can be inserted into the vasculature, musculature, or organ tissue. The sensor may include at least one working electrode along with a counter electrode and a reference electrode. Alternatively, many embodiments utilize at least one working electrode in conjunction with a combined counter/reference electrode.

Embodiments of the analyte monitoring system can be configured to measure analytes such as lactate, ketones, glucose, tissue oxygen and the like. Furthermore, while some embodiments may be configured to measure a single or individual analyte, other embodiments can be configured to measure multiple analytes including various combinations of at least two or more molecules of interest such as lactate, ketone, glucose, oxygen, reactive oxygen species and the like. In preferred embodiments, an infusion cannula along with a single sensor capable of detecting or measuring multiple analytes is inserted or placed within a subject via a single point of entry from a single sharp. In other embodiments, the sensor may be inserted using a first sharp and the infusion cannula may be inserted using a second sharp.

However, it may be beneficial to utilize a single sharp to insert or place both the sensor and infusion cannula simultaneously in order to minimize anxiety and/or discomfort for the user or subject.

The various embodiments discussed below are intended to be exemplary and should not be viewed or construed as discrete individual embodiments. Rather, where possible, individual features or elements discussed in each embodiment should be considered transferable or combinable with the various other embodiments disclosed below.

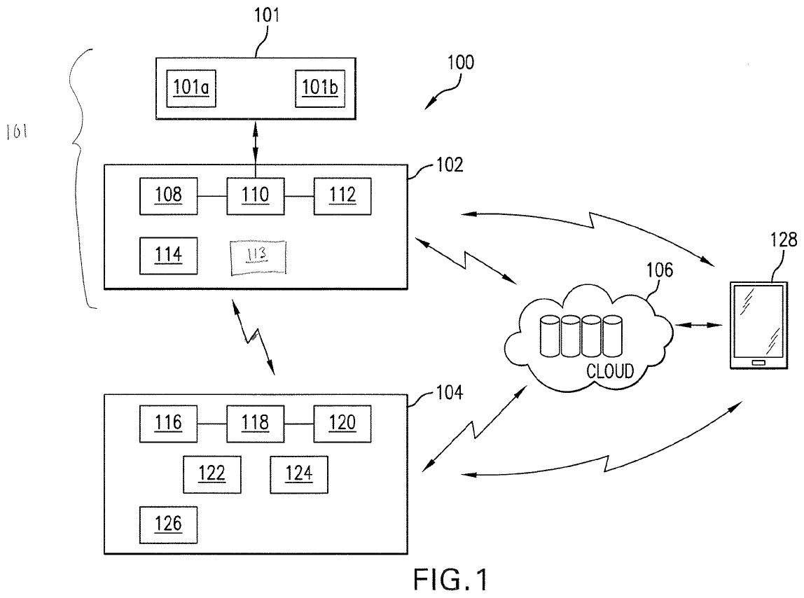

FIG. 1 is an exemplary block diagram showing components within a system 100 for an on-body assembly 101 that enables a single device to monitor the presence of at least one analyte within a subject while further enabling delivery of an infusate to the subject, in accordance with embodiments of the invention. Broadly, the system 100 includes the on-body assembly 101 that includes sensor parts 101a and infusion pump parts 101b that are powered by an electronics module 102 that further enables bi-directional communication with a plurality of remote devices, such as, but not limited to an external monitor 104, cloud computing systems 106 and mobile devices 128. The remote devices enable different aspects of functionality of the system 100, such as, but not limited to entry of patient specific data, display of historical and trending data acquired by the system 100, and machine learning. The totality of components shown in FIG. 1 enables the system 100 to be used across a variety of environments such as triage, patient monitoring and remote monitoring. However, embodiments tailored for a specific environment may not include all of the components shown in FIG. 1. For example, use of the system 100 as a remote monitor in a home may not utilize an external monitor 104. Likewise, for example, when the system 100 is used as a patient monitor in a hospital environment, the system 100 may or may not include a mobile device 128. The inclusion of all of the components within FIG. 1 is intended to illustrate the flexibility and adaptability of the system 100 to be used in different environments. However, regardless of environment, an element of the system 100 that is required for all embodiments is the on-body assembly 101.

As illustrated, the on-body assembly 101 includes sensor parts 101a. In some embodiments the sensor parts 101a include an analyte sensor. In many embodiments the analyte sensor may have various configurations that enable it to detect a single analyte or a plurality of analytes. Exemplary analytes or substances that may be detected or measured using the sensor parts 101a include glucose, lactate, ketones and oxygen or combinations thereof. In preferred embodiments the sensors within the sensor parts 101a are electrochemical sensors. In one embodiment the electrochemical sensor is configured as a two electrode system (i.e, a working electrode and a combined counter/reference electrode). In other embodiments, the electrochemical sensor is configured as a three electrode system (i.e., a working electrode, a counter electrode, and a reference electrode). In embodiments configured to measure a plurality of analytes each different analyte is detected on a separate working electrode. Thus, in embodiments where two different analytes are being measured or detected, the sensor parts 101a will include two working electrodes. Similarly, in embodiments where three different analytes are being measured or detected, the sensor parts 101a may include three working electrodes.

In many embodiments configured to detect or measure multiple different analytes, a combined counter and reference electrode may be shared between multiple working electrodes. For example, an embodiment measuring two analytes may have two working electrodes, and a single, shared, combined counter/reference electrode. Similarly, in embodiments using a three electrode system, two working electrodes measuring different analytes may share a reference electrode and a counter electrode. In still other embodiments each working electrode may have a dedicated combined counter/reference electrode or its own pair or counter electrode and working electrode.

In some embodiments the sensor parts 101a includes a combination of analytes such as, but not limited to, glucose, lactate and oxygen sensors. In other embodiments, the sensor parts 101a includes at least one each of glucose, lactate, and oxygen sensors. In many embodiments, the respective sensors for glucose, lactate and oxygen are electrochemical sensors that include multiple electrodes or transducers that are dispersed along and/or among a sensor assembly.

The sensor parts 101a may further include additional sensors or abilities to measure physical characteristics such as, but not limited to movement (via accelerometers), temperature, tissue impedance (such as tissue hydration levels related to development of edema), skin impedance and sensor hydration levels. The specific physical sensors discussed should not be construed as limiting. Other and additional physical characteristics from physical sensors associated with the sensor parts 101a can be used as inputs to the system 100. Alternatively, in some embodiments, physical sensors associated with components of the system 100, such as, but not limited to the mobile device 128 may provide input to the system 100.

In some of these embodiments two of the three analyte sensor values are combined with data from any to all of the physical characteristic sensors to determine a risk score of developing sepsis. In other embodiments, all three of the analyte sensor values are used in conjunction with the data from any one to all of the physical characteristic sensors to determine a risk score of developing sepsis. The rationale for enabling risk score calculations based on less than all three analytes is to enable tailoring of the sensor array to a particular environment. For example, it may not be necessary to monitor glucose for a septic patient being monitored to determine efficacy of the therapy. Stated another way, for a patient already known to have sepsis, monitoring of glucose may be optional because elevated glucose, as a metric within the system, is used to determine if an infection is progressing toward sepsis. Conversely, in an exemplary remote monitoring application where the objective is to identify early signs of the development of sepsis, the system may be highly tuned to monitor for elevations in glucose accompanied by decreases in tissue oxygen levels and optionally forgo any measurement of lactate.

In many embodiments, the analyte sensors are intended to be placed in subcutaneous tissue where the plurality of working electrodes within the sensor array 101 produce a corresponding plurality of signals. Placement within subcutaneous tissue enables a unique perspective for an oxygen sensor that is substantially different than common SpO2 oxygen measurements. Specifically, with embodiments of the analyte sensors 101a, oxygen within tissue is being measured rather than a measurement of SpO2 which is an estimation of arterial oxygen. When determining a risk score of developing sepsis it is advantageous to measure oxygen within tissue rather than estimated arterial oxygen because oxygen within tissue is a direct measurement of oxygen perfusion which is an early indicator of organ failure.

Supplementing the tissue oxygen signal are signals from the lactate and glucose sensors where each respective signal is proportional to an amount of analyte present in the subcutaneous tissue. In some embodiments, a two-electrode system is employed where each of the working electrodes electrochemically measure a particular analyte relative to a counter electrode. In other embodiments, a three-electrode system is employed where each of the working electrodes electrochemically measure a particular analyte relative to a counter and reference electrode.

For simplicity, the infusion pump parts 101b are illustrated as a single block but may include components such as those necessary to accurately infuse quantities of a medicant such as, but not limited to insulin into a subject. Exemplary non-limiting components that may be encompassed by the infusion pump parts 101b include a fluid reservoir, one or more fluid communication channels, septums, motors, sensors, sharps, a cannula or catheter, and the like. The inclusion of the infusion pump parts 101b may require the use of one or more seals and/or septums to prevent or mitigate fluid leaks from the infusion pump parts 101b.

The system 100 additionally includes an electronics module 102 that provides power for the on-body assembly 101 and enables bidirectional communication with other system components such as, but not limited to the external monitor 104, cloud computing systems 106 or mobile devices 128. Enabling the electronics module 102 to perform such tasks are electronic module components such as, but not limited to a communication module 108, a processor 110, memory 112, and a power supply 114 enclosed within an electronics module case. The electronics module 102 includes additional components, however, the specific components found in FIG. 1 warrant discussion regarding operation of the system 100.

In preferred embodiments the power supply 114 provides power to the electronics module 102 and also to the sensor parts 101a and infusion pump parts 101b. Batteries, rechargeable or disposable, can be used for the power supply 114. In order to minimize the likelihood of fluid ingress to the electronics module, it may be preferable to use inductive charging for embodiments using rechargeable batteries. Other embodiments use alternatives to batteries such as, but not limited to capacitors, supercapacitors, solar cells, fuel cells and the like. The specific examples provided for the power supply 114 should not be construed as limiting. Rather, the examples provided should be viewed as examples of portable power supplies capable of supplying the electronics module 102 and the on-body assembly 101 with power for the expected life of the system 100.

In some embodiments the processor 110 is a custom circuit such as but not limited to an application-specific integrated circuit (ASIC) or field programmable gate array (FPGA). In other embodiments the processor 110 is a more generic system on chip (SoC) or system in package (SiP). In instances where a SoC or SiP is utilized, communication module 108 and memory 112 can be integrated within the SoC or SiP. In many embodiments the processor is in communication with the sensor parts 101a and the infusion pump parts 101b receiving raw signal data from the plurality of working electrodes and other sensors. In some embodiments the processor 110 performs minimal manipulation of the raw data from the working electrodes. Examples of minimal manipulation include, but are not limited to filtering noise and compression. In these embodiments the data from the working electrodes is transmitted to a multitude of external devices via the communication module 108 where processing is completed to monitor an analyte or analytes along with dispensing the infusate via the infusion pump parts 101b. Alternatively, in other embodiments the processor 110 executes stored instructions to process the sensor data before transmitting processed data that may include automated or semi-automated dispensing of the infusate along with transmission of analyte or analytes data to any external devices via the communications module 108.

In many embodiments the communications module 108 is based on personal area network technology commonly referred to as Bluetooth low energy (BLE) or Bluetooth Smart. In other embodiments, the communications module 108 may support other standard wireless communications protocols such as WiFi, and mobile wireless protocols such as 4G, 5G and the like. In still other embodiments, a customized or semi-custom communication standard is utilized. However, one common trait for any communication module 108 is the ability to securely send and receive data between at least a third party device and the electronics communication module 102. The ability to securely transmit either raw or processed data using the communications module 102 enables flexibility that allows the system 100 to be adaptable from a mobile monitor to being an integral component within a hospital ward.

In one embodiment data from the on-body assembly 101 is sent via the communications module 108 to a cloud computing system 106, also commonly referred to as “the cloud”. In still other embodiments data from the on-body assembly 101 is transmitted via the communications module 108 to an external monitor 104. Clinical settings, such as a hospital ward where multiple monitors display a plurality of conditions being monitored for a patient, could be ideal for embodiments where the electronics module 102 transmits to an external monitor 104 or the cloud 106. For example, with the appropriate infrastructure, data from the sensor array 101 can be transmitted in real-time to an electronic medical record stored in the cloud 106. Alternatively, in some embodiments data can be transmitted from the external monitor 104 to the cloud 106 where it is stored as part of an electronic medical record.

In still other embodiments, the electronics module 102 transmits data from the on-body assembly 101 to a mobile device 128 such as, but not limited to a smartphone, a smartwatch, a portable fitness monitor, a tablet, a notebook computer, an ultrabook computer, or an aftermarket or integrated infotainment center for a vehicle. The examples of a mobile device 128 are not intended to be construed as limiting. Rather, the examples are intended to provide guidance regarding the types of devices that can receive and/or transmit data to the electronics module 102. Accordingly, devices that can be viewed as similar to those listed should be considered contemplated by the current disclosure. In embodiments where the mobile device 128 includes a connection to the internet, the mobile device 128 can send data to the cloud 106 where the data can be archived, shared with other devices, be further processed and/or become data to enable machine learning. Utilizing the data to enable machine learning further enables data-driven improvements such as development of algorithms that are patient specific or algorithms that are applied universally across all patients. For example, depending on how much information is provided with the data provided for machine learning, patient specific algorithms can include, but are not limited to factors such as age, race, weight, and pre-existing conditions. Similarly, regardless of patient specific information, all data processed via machine learning can be utilized to improve algorithms with the goal being improved outcomes for all patients.

Additionally, in many embodiments, software being executed on the mobile device 128 may utilize and/or manipulate data from the on-body assembly 101. In still other embodiments, software executed on the mobile device 128 may enable data transmission between the mobile device 128 and the on-body assembly 101 to effectuate modification of operation of the on-body assembly 101.

Even with embodiments where additional processing is handled on either an external monitor 104 or the cloud 106, memory 112 can be used to store data from the sensor array 101 on the electronics module 102. Using the memory 112 to store data from the sensor array 101 can ensure sensor data is not lost if there are connectivity interruptions between the electronics module 102 and the external monitor 104, the cloud 106, or a mobile device 128. The memory 112 can further be used to store program instructions for the processor, or to store values for variables used by the processor 110 to output a risk factor for sepsis.

In many embodiments the electronics module 102 is removably coupled with the sensor parts 101a and the infusion pump parts 101b. With these embodiments, the electronics module 102 is capable of being reused after either the sensor parts 101a or the infusion pump parts 101b are deemed consumed or depleted. In other embodiments, a permanent coupling is achieved after an initial coupling between the electronic module 102 and the sensor parts 101a and the pump parts 101b. In these embodiments, the electronics module 102 is considered disposable and is intended to be discarded after either the sensor parts 101a or the infusion pump parts 101b are deemed consumed. Alternatively, to reduce environmental impact, select portions of the electronics module, such as, but not limited to the power supply 114 and communications module 108 are reusable or recyclable. In many embodiments, initially coupling the electronics module 102 to the sensor array 101 provides power to the electrodes and initiates the program instructions stored in either the processor 110 or the memory 112.

In many of these embodiments, the electronics module 102 includes a feedback device 113. The feedback device 113 provides feedback regarding the status of the combined electronics module 102 and sensor array 101. For example, in some embodiments the feedback device 113 is a single or a plurality of multi-colored LED that blinks a first color and/or first pattern when the system is functioning with design parameters and a second color and/or second pattern if there is an error within the system. In other embodiments, the LED is a single color that uses different frequencies of blinks to convey a status of the system. In still other embodiments, the feedback module includes a vibration device similar to those used in mobile phones to convey status of the system. In still other embodiments, a piezo or other audible sound emitting device is used as the feedback device 113.

The external monitor 104 may include some components not found in the electronics module 102, such as a graphic user interface (GUI) 122 and a display 124. Other components of the external monitor 104, such as a communication module 116, a processor 118, a memory and a power supply 126 may seem duplicative of components in the electronics module 102, but may have different or improved capabilities or functionality. For example, while the power supply 114 of the electronics module 102 may be a battery, the power supply 126 for the external monitor 104 may include an AC power supply that is supplemented with a rechargeable battery to enable the external monitor 104 to operate seamlessly between being plugged into a wall socket and being moved throughout a hospital until it can eventually be plugged back into a wall socket.

For purposes of this invention, the GUI 122 further includes human interface devices that enable interaction with the GUI 122 such as but not limited to virtual or physical keyboards, touchscreens, joysticks, control pads and the like. Accordingly, use of the GUI 122 in conjunction with the display 124 enables user input to the system 100 and further allows selection or customization of what is shown on the display 124. The GUI 122 in conjunction with the communication module 116 and the communication module 108 further enables settings on the electronics module 102 to be manipulated or adjusted to optimize output from the system 100. Similarly, the GUI 122 enables user input to the processor 118 or the memory 120 to enable input and adjust settings for the system 100.

The system 100 further optionally includes a mobile device 128 having a user interface, such as, but not limited to a smartphone, a mobile phone, a smartwatch, a laptop, an ultrabook, a tablet computing device, a pager, and the like. The mobile device 128 is configured to receive data from the electronics module 102 via at least one of the cloud 106, the external monitor 104, or the electronics module 102 itself. In many embodiments the mobile device 128 is in bidirectional communication with the electronics module 102 which enables input via the user interface of the mobile device 128 to be transmitted to the electronic module 102. This enables a user of the mobile device 128 to manipulate, configure, or program settings on the electronics module 102 that control or manage the sensor parts 101a and the infusion pump parts 101b. In some embodiments, bidirectional communications enables processing of data from the sensor array 101 on the mobile device 128. Additionally, in embodiments where the mobile device 128 includes a display, real time data and trends derived from the data is shown on the mobile device 128. In embodiments where the mobile device 128 includes at least one of an audible, tactile, and/or visual alarm, the mobile device 128 can be used to update users of the mobile device 128 of the status of a patient wearing the sensor array 101. The status of the user includes, but is not limited to, whether the system 100 is functioning properly, faults within the system 100, or real time measurements from the sensor array 101.

Another optional component within the system 100 is the cloud 106. Generally, the cloud 106 is considered some type of cloud computing which can be generalized as internet based computing that provides on demand shared computing processing resources and data to computer and other internet connected devices. In some embodiments the cloud 106 receives data from the electronics module 102 directly. In other embodiments data from the electronics module is transmitted to the mobile device 128 before being transmitted to the cloud 106. In still other embodiments, the cloud 106 receives data from the electronics module 102 via the external monitor 104. In still other embodiments, various permutations of communications initiated by the electronics module and transmitted between the external monitor 104 and the mobile device 128 results in data being transmitted to the cloud 106.

Data received by the cloud 106 may have already been processed by an intermediary device or can be processed on or via the cloud 106 and transmitted back to the intermediary device. In some embodiments, the cloud 106 contains electronic medical records and data from the system 100 is automatically uploaded to the electronic medical records. With real time data being uploaded to the cloud, it becomes possible to apply machine learning which can further enable automatic or semi-automatic adjustments to the system 100. Automatic updating the system 100 would result in changes to the programming of the electronics module 102 without human intervention whereas semi-automatic updating would require someone such as a user or medical professional to confirm changes to the programming of the electronics module 102. In one example, the cloud 106 enables examination of medical history such as pre-existing conditions and family history and machine learning can suggest or set customized thresholds and sensor sampling rates based on previous data from patients with similar conditions and data.

The previously discussed components or elements within the system 100 are intended to be exemplary rather than limiting. As the system is intended to be flexible, components are able to be added and removed based on immediate needs. This includes enabling or disabling system components within one environment while enabling or disabling the same system components at a later point. For example, a facility utilizing the system for triage may not implement or enable communications to a mobile device 128 while enabling communication with the cloud 106. However, once a patient is moved from triage to a monitoring or remote monitoring environment, communication with a mobile device may be enabled. Similarly, a facility may choose to not enable communication with the cloud 106.

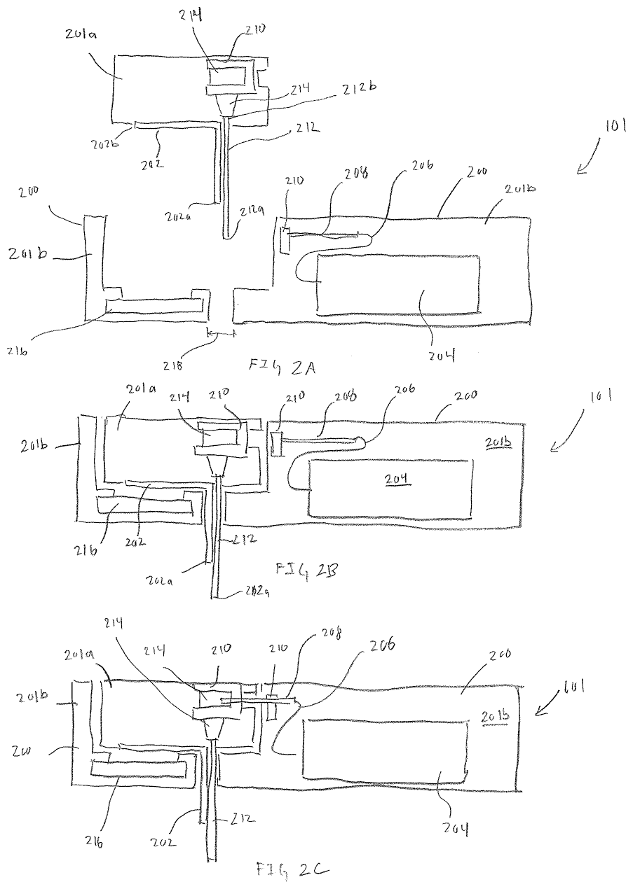

FIGS. 2A-2C are exemplary illustrations of an on-body assembly 101 that includes sensor parts 101a and infusion pump parts 101b, in accordance with embodiments of the present invention. In FIG. 2A the on-body assembly 101 is separated into two parts, a first piece 201a and a second piece 201b. The first piece 201a includes a housing 200 that at least partially contains the following sensor parts 101a: a sensor 202 having a proximal end 202b and a distal end 202a. The distal end 202a of the sensor is intended to be implanted within a subject while the proximal end 202b is intended to interface with the on-body assembly 101. The first piece 201a further includes the following infusion pump parts 101b: a fluid channels 214, septums 210, and a cannula 212 having a distal end 212a and a proximal end 212b. The septum 210 within the first piece 201a prevents fluid leaks between the fluid channels 214 and the cannula.

The distal end 212a is intended to be implanted within a subject and enable infusion of a fluid via the infusion pump parts 101b. In preferred embodiments, the distal end 202a and distal end 212a are in close enough proximity to be inserted or implanted in the subject using a single sharp (not shown). However, in various embodiments, it may be desirable to use two separate sharps; a first sharp to implant the sensor 202 and the second sharp to implant the cannula 212.

The second piece 201b includes a housing 200 that contains a sensor interface 216, at least one septum 210, a member 208, a fluid channel 206 and a pump mechanism 204. For simplicity, it should be understood that the pump mechanism 204 includes a pump motor and a reservoir to contain the fluid infusate. Additionally, the second piece 201b further includes an aperture 218 that enables the distal end 202a and distal end 212a to protrude through the housing 200, into a subject. In many embodiments, the sensor interface 216 within the second piece 201b is a type of circuit board that enables electrical coupling between electronics contained within the housing 200 and the sensor 202. Non-limiting, exemplary sensor interfaces include, but are not limited to flex circuits, printed circuit boards or the like. Additionally, in many embodiments, the member 208 is a needle or sharp that is in fluid communication with the fluid channel 206 to the pump mechanism 204. For simplicity, the components of the electronics module are not illustrated in FIGS. 2A-2C. It should be understood that various components of the electronics module may be split between both the first piece 201a and the second piece 201b. However, when the first piece 201a and the second piece 201b are coupled together, a unified housing 200 for the on-body assembly 101 is formed enabling operation of the sensor parts 101a and the infusion pump parts 101b.

FIG. 2B is a schematic illustrating an exemplary interface between the first piece 201a and the second piece 201b, in accordance with embodiments of the present impression. When the first piece 201a is coupled to the second piece 201b the fluid channel 214 is substantially aligned with the member 208. Furthermore, the sensor 202 is electrically connected or coupled to the sensor interface 216 enabling powering of the sensor 202. In some embodiments, the first piece 201a may include an optional sensor interface (not shown) that retains the sensor 202 to the first piece 201a and includes electrical connectivity to the sensor 202. In these embodiments, coupling or connecting the first piece 201a to the second piece 201b can complete a circuit to the sensor interface 216 to enable powering of the sensor 202.

FIG. 2C is a schematic further illustrating fluid communication between the first piece 201a and the second piece 201b, in accordance with embodiments of the present invention. In this embodiment, the member 208 pierces the septums 210 to place fluid channel 214 in fluid communication with fluid channel 206. In these embodiments, movement of the member 208 may be actuated by an electric motor or may be manually actuated via a user operated slide extending through the housing 200. Accordingly, in these embodiments, the fluid channel 206 may be flexible or include slack that enables the member 208 to move while remaining in fluid communication with the pump mechanism 204. When the member 208 is placed in fluid communication with the fluid channel 214, fluid from the pump mechanism 204 may be dispensed through the cannula into the subject.

In FIGS. 2A-2C, the distal end 212a is illustrated as extending past the distal end 202a. This should not be construed as limiting as other embodiments may have the distal end 202a and the distal end 212a being placed at substantially the same distance from the aperture 218. In still other embodiments, the distal end 202a may be placed so it extends past or beyond the distal end 212a.

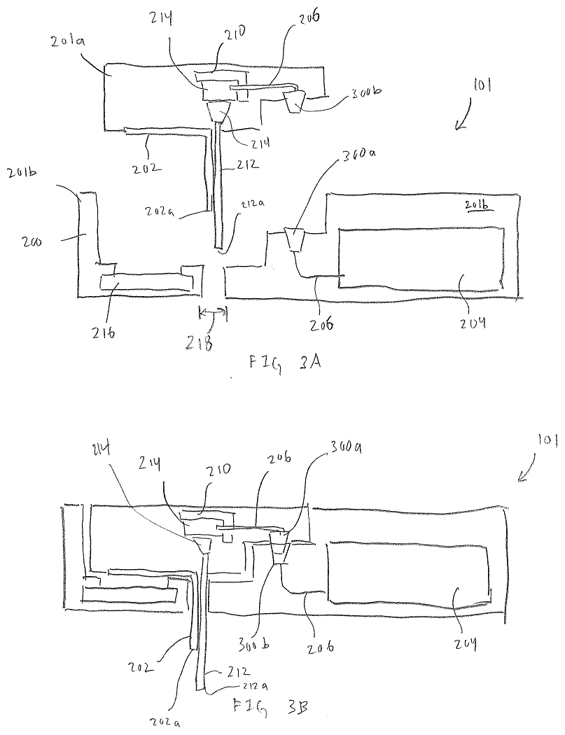

FIGS. 3A and 3B are exemplary illustrations of an alternative on-body assembly 101 that includes sensor parts 101a and infusion pump parts 101b, in accordance with embodiments of the present invention. In FIG. 3A the first piece 201a includes a fluid channel 206 that enables fluid communication between fluid interface 300b and fluid channel 214 (through septum 210). Furthermore, second piece 201b includes fluid interface 300a that is in fluid communication via fluid channel 206 to the pump mechanism 204. It may be advantageous to use fluid interface 300a and 300b because it may further enable infusate to be loaded into a reservoir associated with the pump mechanism 204. FIG. 3B is an exemplary illustration of the first piece 201a being coupled with the second piece 201b thereby coupling fluid interface 300a together with fluid interface 300b. The coupling of fluid interface 300a to 300b places the cannula 212 in fluid communication with the pump mechanism 204 enabling infusate within the pump mechanism 204 to be dispensed through the cannula 212.

The coupling of the first piece 201a and the second piece 201b places or locates the distal end 202a of the sensor 200 and the distal end 212a of the cannula 212 through the aperture 218 and into a subject. In preferred embodiments, the sensor distal end 202a and distal end 212a are inserted through the skin of a subject using a single sharp, such as, but not limited to a needle or custom formed sharp. In other embodiments, it may be preferred to use separate sharps or needles to insert the sensor 200 and the cannula 212. In still other embodiments, the sensor 200 is disposed or built upon a robust conductor such as, but not limited to stainless steel. The use of a stainless steel substrate can enable the sensor itself to pierce or penetrate the skin of a subject to enable percutaneous placement of the sensor 200. Accordingly, in some embodiments the use of a sharp or needle to place or insert the sensor 200 and cannula 212 may be optional.

FIG. 4 is another exemplary illustration of an on-body assembly 101 having a two piece fluid channel 206, in accordance with embodiments of the present invention. The first piece 201a includes a cannula 212 that is coupled to be in fluid communication with partial fluid channel 400a. As discussed above, the first piece 201a further includes the sensor 202. The second piece 201b includes the pump mechanism 204 that is coupled to be in fluid communication with partial fluid channel 400b. As illustrated in detail section A-A, a connector fluid channel 206-1 is formed when partial fluid channel 400a and partial fluid channel 400b are aligned or brought together upon coupling of the first piece 201a to the second piece 201b. In this embodiment the connector fluid channel 206-1 can simplify fluid communication between the first piece 201a and the second piece 201b. In various embodiments, fluid leaks from the connector fluid channel 206-1 formed by coupling partial fluid channels 400a and 400b may be minimized using techniques such as, but not limited to, gaskets, seals, interference fits and/or overlaps between partial fluid channel 400a and partial fluid channel 400b. Additionally, flexible couplings between the partial fluid channel 400a and the cannula 212 may be used to minimize the likelihood of fluid leaks between the partial fluid channel 400a and the cannula 212. Similarly, flexible and/or sealed couplings between the partial fluid channel 400b and the pump mechanism 204 may be used to minimize the likelihood of fluid leakage.

FIG. 5 is an exemplary cross-sectional view looking normal to the page, of a sensor 202 and cannula 212 within a sharp 500 (toward the distal ends 202a/212a), in accordance with embodiments of the present invention. In many embodiments the sharp 500 may be a custom formed sharp having an exterior 502 and an interior 504. The interior 504 of the sharp may be defined as a portion configured to accommodate both the cannula 212 and the sensor 202. As illustrated, the interior 504 is a U-shape that may be formed by folding or bending the sharp.

The sensor 202 has a side A 506a and a side B 506b. In preferred embodiments a working electrode may be formed on either side A 506a or side B 506b. In embodiments having multiple working electrodes, either side A 506a and/or side B 506b may have a single or multiple working electrodes. Likewise, in various embodiments a shared counter/reference electrode for either a single working electrode or multiple working electrodes may be formed on either side A 506a or side B 506b.

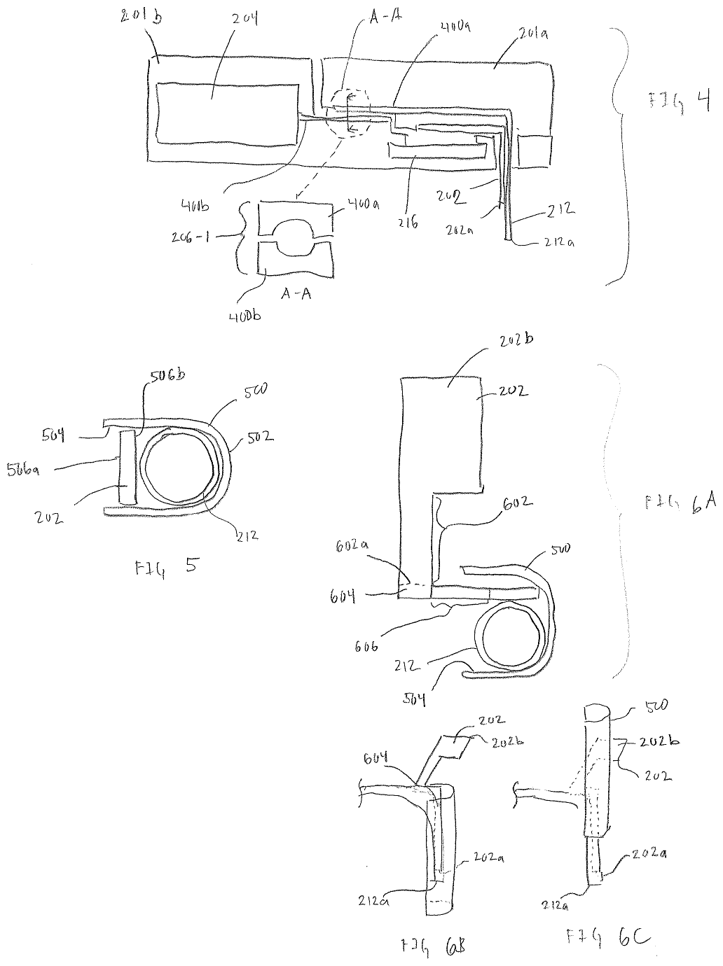

FIGS. 6A-6C are exemplary illustrations of an interface between the sharp 500, the sensor 202, and the cannula 212 during an insertion process, in accordance with embodiments of the present invention. FIG. 6A is a top down view of the sensor 202 relative to a cross-section view of the cannula 212 and the sharp 500. FIG. 6B is an exemplary pseudo-isometric view of the sharp 500 relative to the sensor 202 and the cannula 212 during insertion. FIG. 6C is an exemplary pseudo-isometric view of the sharp 500 being withdrawn over and around the sensor 202 and the cannula 212 after the sensor 202 and the cannula 212 have been inserted into a subject. In FIG. 6A, the proximal end 202b of the sensor 202 is illustrated as being significantly planar with extension 602. The extension 602 includes an extension proximal end 602a. The extension proximal end 602a is the location of a bend 604 in the sensor 202. In FIG. 6A, the bend 604 positions the distal end 202a of the sensor 202 normal to (into) the page (see FIGS. 6B and 6C). FIG. 6A also illustrates sensor jog 606 that places the distal end 202a within the interior 504 of the sharp 500. The cannula 212 is concurrently located within the interior 504 of the sharp 500.

In preferred embodiments electrical contacts for the sensor 202 are located or positioned toward the proximal end 202b. As fluid from either the cannula 212 or bodily fluid from the subject may be wicked along the cannula or sensor, the extension 602 provides a distance or offset from the bend 604 where gaskets or other sealing materials may be placed in contact with the extension 602. Thus, the extension 602 provides an area to seal around the sensor 202 to prevent fluid from reaching the proximal end 202b and reaching the electrical contacts.

As illustrated in FIGS. 6B and 6C, the sensor jog 606 enables placement or location of the sensor 202 within the interior 504 of the sharp 500. Colocating the cannula 212 within the interior 504 of the sharp 500 enables a single insertion to place both the sensor 202 and the cannula 212. The use of a single sharp and single insertion to place both the sensor 202 and the cannula 212 can reduce or minimize anxiety for subjects that may be nervous about using needles to pierce their skin. Additionally, the single insertion point can simplify design of the housing 200 and tools required to perform the insertion. The sensor jog 606 enables the sharp 500 to more fully encompass, or surround, the sensor 202 within the interior 504 of the sharp 500. Covering or enveloping the sensor 202 with the sharp 500, or even nesting the sensor 200 against the cannula 212, can protect or shield side A and/or side B (FIG. 5, element 506a and 506b, respectively) from forces generated by interference between a subject's skin and the sensor 202. Moreover, the sensor jog 606 enables the sharp 500 to be withdrawn around the sensor 202 after insertion, as illustrated in FIG. 6C. Specific routing of tubing or fluid transport to the cannula 212, as illustrated in FIGS. 6B and 6C enables placement of the cannula within the sharp 500 that permits the sharp to be inserted and withdrawn while still being placed around the cannula 212.

The embodiments described above should not be construed as limiting. The sensor array should not be perceived as limited to subcutaneous placement for measurement of glucose, tissue oxygen and lactate. Other embodiments, for use in diagnosing or determining risk score for other conditions or diseases can employ various sensors to measure other combinations of analytes in different locations within the subject.

In many embodiments, additional features or elements can be included, added or substituted for some or all of the exemplary features described above. Alternatively, in other embodiments, fewer features or elements can be included or removed from the exemplary features described above. In still other embodiments, where possible, combinations of elements or features discussed or disclosed incongruously may be combined together in a single embodiment rather than discreetly or in the specific combinations described in the exemplary description found above. Accordingly, while the description above refers to particular embodiments of the invention, it will be understood that many modifications or combinations of the disclosed embodiments may be made without departing from the spirit thereof. The presently disclosed embodiments are therefore to be considered in all respects as illustrative and not restrictive.

1. A system to monitor an analyte and dispense an infusate, comprising:
an on-body assembly having a first piece and a second piece, the first piece containing a sensor and a cannula, the cannula being in fluid communication to a first piece fluid channel, the second piece containing a pump mechanism having a second piece fluid channel,
wherein coupling the first piece to the second piece places the first piece fluid channel in fluid communication with the second piece fluid channel to establish fluid transport from the pump mechanism through the cannula.

2. The system to monitor an analyte and dispense an infusate as described in claim 1, wherein the first piece fluid channel includes a first partial fluid channel.

3. The system to monitor an analyte and dispense an infusate as described in claim 2, wherein the second piece fluid channel includes a second partial fluid channel.

4. The system to monitor an analyte and dispense an infusate as described in claim 3, wherein coupling the first piece to the second piece aligns the first partial fluid channel and the second partial fluid channel and forms a connector fluid channel between the first piece and the second piece.

5. The system to monitor an analyte and dispense an infusate as described in claim 4, further comprising a sharp having an interior, the sharp being sized to accommodate at least a distal end of the sensor and a distal end of the cannula within the interior.

6. The system to monitor an analyte and dispense an infusate as described in claim 5, wherein the distal end of the cannula extends beyond the distal end of the sensor.

7. The system to monitor an analyte and dispense an infusate as described in claim 5, wherein the sensor further includes a proximal end, the distal end of the sensor being offset from the proximal end by a sensor jog.

8. The system to monitor an analyte and dispense an infusate as described in claim 7, wherein the sensor further includes a bend, the bend placing the distal end of the sensor substantially normal to the proximal end of the sensor.

9. The system to monitor an analyte and dispense an infusate as described in claim 8 further comprising an extension located between the proximal end of the sensor and the bend.

10. The system to monitor an analyte and dispense an infusate as described in claim 9, further comprising a seal being positioned across the extension, the seal preventing fluid ingress toward the proximal end of the sensor.

11. A system to monitor an analyte and dispense an infusate, comprising:
an on-body assembly that includes a communication module, a processors, a memory and a power supply, the on-body assembly being comprised of:
a first piece having a sensor and a cannula, the cannula being in fluid communication with a first piece fluid channel, and
a second piece having a pump mechanism being coupled to a second piece fluid channel;
wherein coupling the first piece to the second piece places the first piece fluid channel in fluid communication with the second piece fluid channel to establish fluid communication between the pump mechanism and the cannula.

12. The system to monitor an analyte and dispense an infusate as described in claim 11, wherein the communication module, the processor, the memory and the power supply are distributed between the first piece and the second piece.

13. The system to monitor an analyte and dispense an infusate as described in claim 12, where the first piece fluid channel includes a first partial fluid channel.

14. The system to monitor an analyte and dispense an infusate as described in claim 12, where the second piece fluid channel includes a second part fluid channel.

15. The system to monitor an analyte and dispense an infusate as described in claim 14, wherein coupling the first piece to the second piece aligns the first partial fluid channel and the second partial fluid channel to form a connector fluid channel between the first piece and the second piece.
